(19) United States
(12) Patent Application Publication
(10) Pub. No.: US 2008/0206175 A1
(43) Pub. Date: Aug. 28, 2008

(54) Composition for Skin Whitening and Wrinkle Improvement Comprising Vaccinium Uliginosum Extract and Method for Preparation Thereof

(76) Inventor: Se Young Chung, Seoul (KR)

Correspondence Address: KED & ASSOCIATES, LLP, P.O. Box 221200, Chantilly, VA, 20153-1200, US

(21) Appl. No.: 11/813,644

(22) PCT Filed: Jan. 11, 2006
(86) PCT No.: PCT/KR2006/000108

§ 371 Date: Mar. 14, 2008

(30) Foreign Application Priority Data
Jan. 11, 2005 (KR) 10-2005-0002473
Jan. 11, 2005 (KR) 10-2005-0002474

(51) Int. Cl. A61K 8/97; A61Q 19/02

(52) U.S. Cl. 424/62

(57) ABSTRACT

The present invention relates to a composition for the skin whitening and wrinkle improvement, comprising an extract of Vaccinium uliginosum as an active ingredient. The Vaccinium uliginosum extract has the effects of inhibiting and scavenging reactive oxygen species produced in skin tissue as a result of ultraviolet irradiation to the skin, effectively inhibiting tyrosinase activity to inhibit the production of melanin in melanin cells, inhibiting the secretion of cytokines in keratinocytes, promoting the production of procollagen, and inhibiting the decomposition of collagen. Thus, the extract will be useful for the prevention of skin photo aging caused by ultraviolet radiation, the enhancement of skin whitening and the improvement of wrinkles. In addition, the Vaccinium uliginosum extract is suitable to use as a component for improving skin wrinkles in cosmetics, foods and drugs, because the Vaccinium uliginosum extract can achieve the effect of improving skin conditions, even when it is applied to the skin or used internally.

### TECHNICAL FIELD

The present invention relates to a composition for skin whitening and wrinkle improvement, containing a Vaccinium uliginosum extract, and more particularly to a composition for the improvement of skin conditions, which can prevent and improve skin discoloration, freckles, pigmentation, etc., to enhance skin whitening, can prevent and improve skin wrinkles, and can enhance skin firmness. The inventive composition for the improvement of skin conditions can be easily prepared in the form of an extract or dried extract powder, and can be used as one component of cosmetics, health functional foods, drugs, etc, for the improvement of skin conditions.

### BACKGROUND ART

With a recent increase in aging population resulting from an increase in average lifespan, studies on aging in the medical, biological and food fields are gradually increasing. All living organisms are aged as they grow older, in which the aging means that the ability of organisms to adapt to environmental changes is gradually reduced with the passage of time.

The aging likewise occurs in the skin. Deteriorations in skin conditions, such as the occurrence of skin wrinkles and pigmentation and a reduction in skin firmness, are main phenomena resulting from skin aging, and the skin undergoes an aging process by various structural changes caused by various factors occurring in the internal and external environments of the skin. However, modern persons hope to make the skin more clean and beautiful, and various studies and experiments on methods and materials for improving skin conditions (preventing skin aging) are also being actively conducted.

The causes of skin aging, a main phenomenon making ski conditions worse, can be broadly divided into intrinsic aging and extrinsic aging. The intrinsic aging occurs with an increase in age, and the extrinsic aging is caused by external factors, such as ultraviolet radiation. Particularly the extrinsic aging is also called “photo aging” since it progresses mainly by ultraviolet radiation. Characteristic clinical skin findings observed in the intrinsic skin aging process include fine wrinkles, dermal atrophy, and the reduction of a subcutaneous fat layer. In a photo aging process by sunlight's ultraviolet radiation, which forms the largest portion of the extrinsic aging, reactive oxygen species (ROS) are excessively produced in the skin epidermis by ultraviolet radiation, in which such reactive active species cause pigmentation resulting from an increase in melanin, suppress the biosynthesis of collagen and elastin in the skin and induce the stimulation of decomposition of collagen and elastin, thus forming wrinkles. In particular, the present invention relates to the improvement of skin conditions (skin whitening, wrinkles, etc.) undergoing the photo aging process.

Although the action mechanism of photo aging has not yet been clearly established, it is known through various studies that ultraviolet radiation causes the modification of nucleic acid and protein and the oxidation of lipid to cause cell chromosomal modification and cell membrane injury, or mediates reactive oxygen species to cause cell modification. Also, ultraviolet irradiation from the sun causes inflammatory reactions, such as erythema and edema, and various clinical changes, such as skin darkening and the modification of a substance between dermal cells. Moreover, studies to identify whether any of such many reactions has a deep connection with skin wrinkles, skin firmness, etc., are ongoing.

Typical phenomena which can occur as skin conditions become worse due to photo aging caused by ultraviolet radiation, natural aging and the like, include a phenomenon where skin color becomes black and dark. Pigments associated with skin color include melanin, melanoid, carotene, oxygenated hemoglobin and reduced hemoglobin, the most important being melanin. Melanin functions as a camouflage means for self-protection and absorbs or scatters ultraviolet radiation to prevent cells or tissues in the cells from being injured by ultraviolet radiation. Melanin has no specific peak absorbance wavelength, absorbs light at the entire wavelength range, and also has excellent function to remove reactive oxygen species, such as superoxide anion, hydrogen peroxide, hydroxyl radicals, and singlet oxygen, from the skin.

However, when melanin is excessively present in skin tissues, melanin will generate reactive oxygen by itself, and in some cases, reduce or oxidize other substances by catechol or quinone in the melanin structure. Also, it is known that melanin itself shows free radical properties such that it forms discoloration, freckles, etc., on the human body to make the skin black and dark, accelerates skin aging, and is involved in the induction of skin cancer.

Those known as melanin production pathways include a chemical pathway where melanin is produced from tyrosine via DOPA and DOPA-quinone by tyrosinase, or a pathway where melanin is produced by migration from melanocytes to keratinocytes.

Known methods for skin whitening by the inhibition of melanin production include a method of shielding ultraviolet radiation, a method of inhibiting the synthesis of core carbohydrates necessary for tyrosinase activity, a method of inhibiting the activity of tyrosinase that is an enzyme associated with melanin formation, a method of interfering with the cleavage of melanin cells using a toxic substance specific for melanin cells, and a method of using vitamin C derivatives and placenta extract.

Japanese Patent Laid-Open Publication No. H6-192062 discloses hydroquinone as a whitening substance. The disclosed hydroquinone shows excellent whitening effect, but has a problem in that it is a carcinogenic substance which is unsuitable for use as the material of cosmetics and the like. Japanese Patent Laid-Open Publication No. S56-7710 discloses kojic acid as a whitening substance. The kojic acid shows excellent ability to inhibit tyrosinase, leading to excellent whitening effect, but has a problem in that it is unsuitable for use as the material of cosmetics, foods, etc., due to the problem of toxicity. Japanese Patent Laid-Open Publication No. H4-9315 discloses arbutin as a whitening substance, which is obtained by extraction or synthesis from natural plant Bearberry inhabits alpine regions. However, the arbutin has a problem in that it causes skin irritation. Also, natural substances, such as Job's tears and cucumbers, have been used long time ago, but these have no connection with the excessive production of melanin.

A phenomenon where skin color becomes black and dark, and also a phenomenon where skin epidermis is damaged and wrinkles occur, are typical phenomena occurring as skin conditions become worse due to photo aging, etc. It is known that, since photo aging is generally prominent in dermal changes, the occurrence of wrinkles is also attributable to dermal changes. Particularly, the prominent changes in the skin's dermal layer are that amorphous firm tissue is excessively accumulated in the outer dermis and that dermal collagen fiber is reduced.

Although it is still difficult to clearly understand the wrinkling process, there are numbers of results associated with wrinkling, including a decrease in dermal collagen synthesis, an increase in the decomposition activity of dermal collagen, the damage of the epidermal basal membrane, and a reduction in epidermal metabolic activity. Also, it is believed that the occurrence of wrinkles results from the overall effects of various biochemical and clinical changes induced by ultraviolet radiation.

In a prior attempt to solve the problem of skin wrinkles, there is the case of making cosmetic products containing collagen. However, if collagen is applied to the skin surface in the form of cosmetics, there is a problem in that the transdermal absorption of the polymer collagen is difficult, and so the function thereof cannot be sufficiently expected. Moreover, there is a method of injecting collagen directly into the skin dermis, but this method is not regarded as a solution to improve skin wrinkles, due to side effects.

Substances known to stimulate collagen synthesis include retinoic acid, and an animal placenta-derived protein (Japanese Patent Laid-Open Publication No. H8-231370). Retinoic acid requires complex technology for formulation and has limitations in use in terms of safety, since it causes, e.g., skin irritation. The animal placenta-derived protein has a fetal problem in that an extract from cattle attacked with bovine spongiform encephalopathy can be used. Also, alpha-hydroxy acid (ABA) confirmed to be effective in the human body, and various vitamin A derivatives (retinoids), have been developed and used in cosmetics. However, those having secured clinical effects proved so far are only said substances and Uv screening agents. From the 1990s in Europe, wrinkle improvement effects have already been written and advertised in cosmetics, and around the year 1993, components for improving skin conditions, such as ceramide, AHA and retinol, were introduced in cosmetics, and the new term “functional cosmetics” was made.

Almost all of cosmetic companies have developed cosmetics for skin whitening or wrinkle improvement, but these products were limited to cosmetics and could not achieve the effect of improving skin wrinkles by ingestion. Also, considering that “eating cosmetics” have a faster effect than that of “application cosmetics”, there is an urgent need for the research and development of “eating cosmetics for the improvement of skin conditions”, as well as “functional food” for improving skin conditions”.

Materials reported in the art to have the effect of improving skin conditions by ingestion include very limited kinds of skin whitening materials, such as vitamin C, vitamin E, and guava extract. Even in the case of application cosmetics, the materials are limited only to hydroquinone and stabilized derivatives of arbutin, kojic acid and vitamin C, which are thought to be precursors of hydroquinone, as well as natural substances (cytokine regulation associated with melanin synthesis). In the case of application cosmetics, the effects of these compounds have been verified by a variety of in vitro tests, but since these cosmetics do not give high satisfaction, such as a feel for other wrinkle-improving and moisturizing products, numerous derivatives are still synthesized, and the effects of novel natural substances on the improvement of skin conditions are examined. However, the development of novel products for, e.g., oral administration, is still far distant.

Meanwhile, Vaccinium uliginosum used for the first time as a component for the improvement of skin wrinkles in the present invention is a deciduous shrub belonging to the Rhododendron family, which is a plant that grows naturally in Halla Mountain, Geumgang Mountain, Baekdu Mountain, etc., of the Korean Peninsula, flowers in June to July and bears fruit in August. Components contained in Vaccinium uliginosum may include saccharides (8-11.8%), fruit acid (2-2.25%), tannic acid (0.15-0.25%), and cellulose. The pharmacological actions of Vaccinium uliginosum, which have been known so far, may include vascular protection, dysentery treatment, antiulcer, anticancer, the treatment of diabetic retinal disease, the prevention of geriatric diseases, postpartum recovery, blood purification, urination, and the treatment of rheumatoid arthritis. However, the effects of Vaccinium uliginosum on skin wrinkle improvement and skin whitening are not yet known.

### DISCLOSURE OF THE INVENTION

### Technical Problem

It is an object of the present invention to provide a composition for skin whitening and wrinkle improvement which contains an extract of Vaccinium uliginosum as an active ingredient and a preparation method thereof.

It is another object of the present invention to provide a cosmetic composition, food composition and pharmaceutical composition containing a Vaccinium uliginosum extract, and the use thereof as an agent for skin whitening and wrinkle improvement.

### Technical Solution

The present invention is based on a finding that a Vaccinium uliginosum extract has an antioxidant effect of inhibiting the production of reactive oxygen species or scavenging the reactive oxygen species, which are the important factors of causing photo aging. When the skin is exposed to ultraviolet radiation, reactive oxygen species, such as superoxide radicals, hydroxyl radicals, hydrogen peroxide and singlet oxygen radicals, will be produced in keratinocytes at high concentrations. It was found that, when the inventive Vaccinium uliginosum extract was administered to skin tissue exposed to ultraviolet radiation, the production of the reactive oxygen species would be significantly reduced.

Also, the present inventors have newly found that the Vaccinium uliginosum extract shows the effect of inhibiting the production of melanin by suppressing tyrosinase activity mediating melanin synthesis and that it shows the effects of increasing the synthesis of collagen in skin fibroblasts, inhibiting the decomposition of collagen and inhibiting the secretion of cytokines in keratinocytes. On the basis of the findings, the present inventors have suggested the novel uses of the Vaccinium uliginosum extract for skin whitening and wrinkle improvement.

The Vaccinium uliginosum extract, a natural material used as a wrinkle improving and skin whitening agent in the present invention, has no particular side effects, and so is highly suitable to prevent and improve skin wrinkles and to enhance skin firmness. Also, the Vaccinium uliginosum extract can sufficiently achieve the effects of whitening the skin and improving skin conditions, such as wrinkles, even when it is applied to the skin or applied internally.

Hereinafter, the present invention will be described in detail with respect to a composition for the skin whitening and wrinkle improvement, a preparation method thereof and the concrete use embodiments thereof.

The inventive composition for the improvement of skin conditions contains the Vaccinium uliginosum extract as an active ingredient. In addition to the Vaccinium uliginosum extract, the inventive composition may further comprise, e.g., various additives and stabilizers, depending on required formulations. The Vaccinium uliginosum extract is obtained by extraction from the fruit, leaf or bark of Vaccinium uliginosum, in which an extraction solvent, such as water or alcohol, is preferably used.

Although particular limitations are not imposed on a preparation method of the Vaccinium uliginosum extract that is a main component used in the present invention, a preferred method for preparing the Vaccinium uliginosum extract according to present invention is as follows.

First, the fruits and/or leaves of Vaccinium uliginosum are washed and extracted using water as a solvent to obtain an undiluted extract {step (a)}. More specifically, the water solvent is preferably used in an amount of 800-1200 ml relative to 100 g of the fruits of Vaccinium uliginosum, and the extraction is preferably performed by heating the plant in a water bath at a temperature of 40-100° C. for 10-15 hours.

Then, the Vaccinium uliginosum extract obtained in the step (a) is filtered and the supernatant is collected {step (b)}. For example, the Vaccinium uliginosum extract is preferably filtered through multi-layer gauze to obtain a supernatant solution from which foreign matter has been removed.

Although the inventive effect of improving skin conditions can be sufficiently achieved only with the Vaccinium uliginosum extract obtained in the step (a) or step (b), the following additional step is preferably performed.

In the next step, the solvent contained in the supernatant obtained in the step (b) is evaporated to concentrate the Vaccinium uliginosum extract, thus obtaining a highly concentrated Vaccinium uliginosum extract {step (c)}. Preferably, the supernatants obtained by repeating the step (a) and step (b) three times are combined with each other, and water contained in the combined supernatant is completely evaporated by means of a rotary evaporator so as to concentrate the Vaccinium uliginosum extract.

Also, following the step (c), the concentrated Vaccinium uliginosum extract is dissolved in a small amount of distilled water and then freeze-dried or spray-dried, such that the Vaccinium uliginosum extract can be used in the form of powder {step (d)}.

As the solvent for extracting Vaccinium uliginosum in the first step, alcohol, such as methanol, ethanol, isopropanol or butanol, in addition to water, can be used. In this case, the fruit or leaf of Vaccinium uliginosum is extracted in alcohol at a temperature of 20-90° C., or sonicated. Alternatively, it may also be extracted by percolation at room temperature or 4° C.

The concrete use embodiments of the inventive Vaccinium uliginosum extract include a cosmetic composition for the improvement of skin conditions, food or health functional food, and a pharmaceutical composition, which will be described in detail below.

The Vaccinium uliginosum extract according to the present invention can be used as an agent for improving skin conditions (e.g., whitening and wrinkles) in the existing cosmetics, and there is no particular limitation on the formulation of the cosmetics. When the Vaccinium uliginosum extract is used to prepare cosmetics, components conventionally used in cosmetics, e.g., conventional adjuvant and carrier components, such as an antioxidant, a stabilizer, a solubilizer, vitamin, a pigment and a fragrance, may be used in addition to the Vaccinium uliginosum extract. Examples of cosmetic formulations include solution, suspension, emulsion, paste, gel, cream, lotion, powder, soap, surfactant-containing cleansing oil, powder foundation, emulsion foundation, and spray, and any person skilled in the art may select and use a suitable carrier depending on the kind of a formulation.

It is preferable in terms of whitening effect that the cosmetic composition should contain at least one component selected from the group consisting of arbutin, kojic acid, Broussonetia extract, 3-ethoxy ascorbic acid, licorice extract and a mixture thereof. Moreover, the cosmetic composition may additionally contain at least one additive selected from the group consisting of retinol, retinol palmitate, polyethoxylated retinamide, adenosine, kinetin, cocoon extract, isoflavon and a mixture thereof.

The dry content of the Vaccinium uliginosum extract is preferably 0.0001-10 wt % based on the total weight of the cosmetic composition. If the content of the Vaccinium uliginosum extract is less than 0.0001 wt %, the effect of wrinkle improvement will be insufficient, and if it is more than 10 wt %, it will not be easily dissolved. Also, increased effects on the inhibition of tyrosinase activity and an increase in the synthesis of collagen, which result from an increase in the Vaccinium uliginosum extract content, cannot be expected, and an increase in raw material cost will be caused.

In another use embodiment of the Vaccinium uliginosum extract, the present invention provides a food for the improvement of skin conditions (e.g., whitening and wrinkles), which contains the Vaccinium uliginosum extract and food additives.

As used herein, the phrase “food for the improvement of skin conditions” is meant to include not only general food, but also “health supplement food” or “health functional food”. The term “health functional food” refers to food that can meet the requirement of food in the form of, e.g., tablets, capsules, powders, granules, liquids and pills, which are prepared and processed from raw materials or components having functionality useful for the human body (Act 3 (1) of a law on health functional food, which is Korean Law No. 7428). As used herein, the term “functionality” refers to obtaining c useful for health applications, such as either controlling nutrients with respect to the structure and function of the human body or physiological action. Namely, it means that food is useful for the health preservation of healthy persons or semi-healthy persons.

The effect of improving skin conditions can be sufficiently obtained, even when a food containing the Vaccinium uliginosum extract is ingested or applied to the skin. However, it is preferable in view of the convenience of administration that the inventive extract be used in the form of functional foods having formulations, such as tablets, sugar-coated tablets, capsules, and drinks.

Other forms of the food for the improvement of skin conditions include beverages, alcoholic drinks, kimchi, yogurt, milk, ice cream, bread, rice cake and noodles. As used herein, the term “food additives” refers to additives used in food by, e.g., addition, mixing and impregnation, in the preparation, processing and preservation of the food.

In another embodiment, the present invention provides a pharmaceutical composition for the improvement of skin conditions, which comprises the Vaccinium uliginosum extract together with a pharmaceutically acceptable carrier. The Vaccinium uliginosum extract has antioxidant function, and shows the effects of not only improving skin wrinkles caused by ultraviolet radiation, such as stimulating the synthesis of collagen and inhibiting the decomposition of collagen, and but also inhibiting tyrosinase activity. This will be clearly understood by Examples as described below.

Suitable formulations of the pharmaceutical composition include, but are not limited to, tablets, sugar-coated tablets, hard or soft capsules, solutions, suspensions, emulsions, injections and suppositories. The kind of the carrier can be easily selected by a person skilled in the art depending on the formulation of the pharmaceutical composition, and may contain at least one component capable of acting as a diluent, a fragrance, a solubilizer, a lubricant, a suspending agent, a binder and a disintegrant.

The dosage of the extract for stimulating the synthesis of collagen, which contains the Vaccinium uliginosum extract, may vary depending on the need of a patient, a condition to be treated and the kind of a compound to be used, and the inventive extract does no cause the problem of side effects, even when it is administered in excess. It is usually preferable that the dosage of the Vaccinium uliginosum extract be 0.001-0.10 g/kg of the patient's bodyweight, based on dry powder.

Hereinafter, a skin whitening process and a wrinkle formation process will be first described in connection with the skin condition improvement effect of the Vaccinium uliginosum extract adopted in the present invention, and then any change in factors associated with skin wrinkles, which can occur when the Vaccinium uliginosum extract is used, will be described by Examples and Test Examples below.

When ultraviolet light from sunlight, which is the main cause of skin aging, reaches the skin, reactive oxygen species will be generated in the epidermal tissue of the skin. The generated reactive oxygen species cause damage to epidermal tissue and stimulate keratinocytes in the epidermal tissue to secrete not only interleukins, such as IL-1α, IL-1β and IL-6, but also cytokines, such as colony stimulating factor and tumor necrosis factor (TNF)-α, in which the secreted interleukins or cytokines affect skin cells to induce complex inflammatory reactions and immune reactions. Also, the reactive oxygen species increase the transfer of melanosome from melanocytes to keratinocytes, and increase the production of melanin in melanocytes, and also inhibit the synthesis of collagen in dermal fibroblasts. These phenomena are very important in the photo-aging process.

When keratinocytes are stimulated with external ultraviolet radiation, they will secrete inflammatory cytokines and the like to promote the proliferation of melanocytes and the biosynthesis of melanin, thus regulating various factors in the growth and formation of melanocytes and the secretion and differentiation of melanin. Also, ultraviolet radiation irradiated into skin tissue stimulates melanocytes in the skin to secrete IL-1α, and the secreted IL-1α again stimulates melanocytes to secrete ET (endothelin)-1. The secreted ET-1 activates protein kinase C and the adenylate cyclase system to induce the proliferation of melanocytes, and promotes tyrosinase activity, thus causing pigmentation.

Also, the above interleukins produced and secreted in keratinocytes stimulate the gene expression of matrix-degrading enzymes, such as matrix metalloproteinase (MMP)-1 (collagenase), MMP-3 (stromelysin-1) and MMP-9 (92-kd gelatinase) to increase the production of MMP, and they suppress the expression of procollagen to reduce the biosynthesis of procollagen. The MMP-1 (collagenase) acts to promote the decomposition of collagen converted from type I procollagen. Namely, when ultraviolet radiation reaches the skin, the MMP-1 will reduce the production of type I procollagen and induce the decomposition of produced collagen to reduce the amount of collagen on the skin. According to this process, wrinkles are formed on the skin.

Effects resulting from the administration of the Vaccinium uliginosum extract will be described by Test Examples below, in connection with the above-described factors associated with skin whitening and wrinkles.

### ADVANTAGEOUS EFFECTS

The inventive composition for skin whitening and wrinkle improvement, which contains the Vaccinium uliginosum extract, inhibits and scavenges reactive oxygen species which are produced in skin tissue as the skin is irradiated with ultraviolet radiation. Also, the inventive composition effectively suppresses tyrosinase activity to inhibit the production of melanin in melanin cells, and suppresses the secretion of cytokines in keratinocytes, promotes the production of procollagen, and inhibits the decomposition of collagen. Accordingly, the inventive composition is useful to prevent the photo-aging of the skin, caused by ultraviolet radiation, and to enhance skin whitening and to improve wrinkle conditions.

### BRIEF DESCRIPTION OF THE DRAWINGS

FIG. 1 is a graphic diagram showing DPPH radical scavenger activity of Vaccinium uliginosum L. extract.

FIG. 2 is a graphic diagram showing superoxide radical scavenger activity of Vaccinium uliginosum L. extract in the xanthine-xanthine oxidase system.

FIG. 3 is a graphic diagram showing superoxide radical scavenger activity of Vaccinium uliginosum L. extract in the NADH/PMS system.

FIG. 4 is a graphic diagram showing hydroxyl radical scavenger activity of Vaccinium uliginosum L. extract.

FIG. 5 is a graphic diagram showing singlet oxygen radical scavenger activity of Vaccinium uliginosum L. extract.

FIG. 6 is a graphic diagram showing superoxide radical from keratinocyte treated with extracts of Vaccinium uliginosum L. after UV B irradiation.

FIG. 7 is a graphic diagram showing hydroxyl radical from keratinocyte treated with extracts of Vaccinium uliginosum L. after UV B irradiation.

FIG. 8 is a graphic diagram showing hydrogen peroxide radical from keratinocyte treated with extracts of Vaccinium uliginosum L. after UV B irradiation.

FIG. 9 is a graphic diagram showing singlet oxygen radical from keratinocyte treated with extracts of Vaccinium uliginosum L. after UV B irradiation.

FIG. 10 is a graphic diagram showing IL-1β release from keratinocyte treated with extracts of Vaccinium uliginosum L. after UV B irradiation.

FIG. 11 is a graphic diagram showing IL-6 release from keratinocyte treated with extracts of Vaccinium uliginosum L. after UV B irradiation.

FIG. 12 shows Type I Procollagen concentration of human dermal Fibroblast treated with IL-1β and extracts of Vaccinium uliginosum L.

FIG. 13 shows MMP-1 concentration of human dermal Fibroblast treated with IL-1β and Vaccinium uliginosum L. extracts

FIG. 14 is a photograph showing the skin replica of a hairless mouse which has been irradiated with ultraviolet radiation on the skin and administered with Vaccinium uliginosum L. extracts.

FIGS. 15(a) to 15(d) show H-R values measured for a hairless mouse which has been irradiated with ultraviolet radiation on the skin and administered with Vaccinium uliginosum L. extracts.

FIG. 16 shows melanin concentration in B 16 melanoma cells of skin tissue, when the skin has been administered with each of Vaccinium uliginosum L. extracts and kojic acid.

FIG. 17 shows melanin concentration in melanoma cells of skin tissue, when the skin has been administered with each of Vaccinium uliginosum L. extracts and kojic acid after UV B irradiation.

### BEST MODE FOR CARRYING OUT THE INVENTION

### Preparation Example 1

### Preparation of Vaccinium uliginosum L. Extracts

100 g of the fruit of Vaccinium uliginosum collected in Bukhan Mountain, Korea, together with 500 ml of 80% water, was heated in a water bath at 50° C. for 12 hours to obtain a Vaccinium uliginosum extract. The Vaccinium uliginosum extract was filtered through multi-layer gauze and the supernatant was collected. The supernatants obtained by repeating the extraction and filtration processes three times were combined with each other, and water contained in the combined supernatant was completely evaporated with a rotary evaporator under reduced pressure to obtain a concentrated hot-water extract.

### Preparation Example 2

### Preparation of Vaccinium uliginosum Extract Powder

The concentrated Vaccinium uliginosum extract was dissolved in distilled water and then spray-dried, thus preparing a final Vaccinium uliginosum extract in the form of powder.

### Preparation Example 3

### Preparation of Vaccinium uliginosum Extract

100 g of the fruit of Vaccinium uliginosum was added to 500 ml of 80% methanol (methanol:water=4:1) and sonicated four times at room temperature to obtain an extract. The extract was filtered through gauze and then the supernatant was collected. The supernatants obtained by repeating the extraction and filtration processes three times were combined with each other, and methanol contained in the combined supernatant was evaporated with a rotary evaporator under reduced pressure. The remaining extract was dissolved in a small amount of distilled water to obtain an alcohol extract of Vaccinium uliginosum.

### Example 1

### Preparation of Skin Lotion for Skin Whitening and Wrinkle Improvement

Using the Vaccinium uliginosum extract (liquid phase) prepared in Preparation Example 1, skin lotion was prepared according to a conventional method. The components and contents of the skin lotion are shown in Table 1 below.

TABLE 1

| Components | Contents (wt %) |
|---|---|
| Vaccinium uliginosum extract | 0.5 |
| Glycerin | 5 |
| 1,3-butyleneglycol | 3 |
| Ethanol | 10 |
| Sodium hyaluronate | 5 |
| PEG 1500 | 1 |
| Alantoin | 0.1 |
| Benzophenone | 0.04 |
| Octyldodeces-16 | 0.2 |
| Polysorbate | 0.2 |
| Distilled water | Balance |
| Sum | 100 |

### Example 2

### Preparation of Lotion for Skin Whitening and Wrinkle Improvement

Using the Vaccinium uliginosum extract (liquid phase) prepared in Preparation Example 1, lotion was prepared according to a conventional method. The components and contents of the lotion are shown in Table 2 below.

TABLE 2

| Components | Contents (wt %) |
|---|---|
| Vaccinium uliginosum extract | 1 |
| Glycerin | 5 |
| 1,3-butyleneglycol | 3 |
| Sodium hyaluronate | 5 |
| Squalan | 5 |
| Glyceryl stearate | 1.5 |
| Stearyl alcohol | 1.5 |
| Polysorbate 60 | 1.5 |
| Lanolin | 1 |
| Sorbitan stearate | 0.7 |
| Trioctanoin | 1.5 |
| Dimethicone | 1 |
| Fragrance | 0.01 |
| Distilled water | Balance |
| Sum | 100 |

### Example 3

### Preparation of Functional Food (Tablet) for Skin Whitening and Wrinkle Improvement

5 mg of the Vaccinium uliginosum extract (powder) prepared in Preparation Example 2 was mixed with 150 mg of lactose BP, 30 mg of starch and 15 mg of pregelatinized corn starch BP. Then, a suitable amount of purified water was added thereto and the mixture was granulated into powder. The granule was dried, mixed with 1 mg of magnesium stearate and compressed to obtain a tablet.

### Example 4

### Preparation of Functional Food (Beverage) for Skin Whitening and Wrinkle Improvement

A composition comprising a functional beverage base containing 2 mg of the Vaccinium uliginosum extract prepared in Preparation Example 1, 5 mg of a food coloring agent, 5 mg of orange essence, 700 mg of fructose, 10 mg of citric acid and 5 mg of vitamin, to which purified water was then added, thus preparing a beverage.

### Example 5

### Preparation of Functional Food (Syrup) for Skin Whitening and Wrinkle Improvement

637.5 g of white sugar was dissolved in 500 ml of purified water. In a separate container, 2.0 g of sodium carboxymethylcellulose was dissolved in 400 ml of purified solution, and then mixed with the white sugar solution. To the mixture solution, 0.28 g of methyl parabene and 0.12 g of propylene parabene were added and dissolved, to which 20 ml of ethanol was added, and purified water was added thereto to make an overall solution volume of 1000 ml. In the solution, the sieved Vaccinium uliginosum extract prepared in Preparation Example 1 was suspended to obtain syrup.

### Example 6

### Preparation of Ointment

5 g of the Vaccinium uliginosum extract prepared in Preparation Example 1 was mixed with 20 g of cetyl palmitate, 40 g of cetanol, 40 g of stearyl alcohol, 80 g of isopropyl myristate, 20 g of sorbitan monostearate, 60 g of polysorbate, 1 g of propyl paraoxybenzoate, 1 g of paraoxybenzoate and a suitable amount of purified water, thus preparing an ointment.

### Example 7

### Preparation of Functional Alcoholic Drink

Deodorized and purified alcohol was diluted in distilled water at a concentration of 40 wt %, to which the Vaccinium uliginosum extract prepared in Preparation Example 3 was then added in an amount of 0.05 parts by weight based on 100 parts by weight of the diluted alcohol solution. To the solution, suitable amounts of stevioside, high fructose, amino acid, citric acid and salt were added, thus preparing a functional alcoholic drink containing the Vaccinium uliginosum extract.

### Test Example 1

### Reactive Oxygen Specie (ROS) Scavenger Activity

1. Measurement Of DPPH Radical Scavenger Activity

0.8 ml of a solution of 1 mM DPPH (2,2-diphenyl-1-picrylhydrazyl) in ethyl alcohol was mixed with 0.2 ml of the Vaccinium uliginosum extract prepared in Preparation Example 1, and the mixture was left to stand at 37° C. for 30 minutes and then measured for absorbance at 517 nm. As a control drug, ascorbic acid (vitamin C) was used. The results were expressed as percentages relative to the group untreated with the sample.

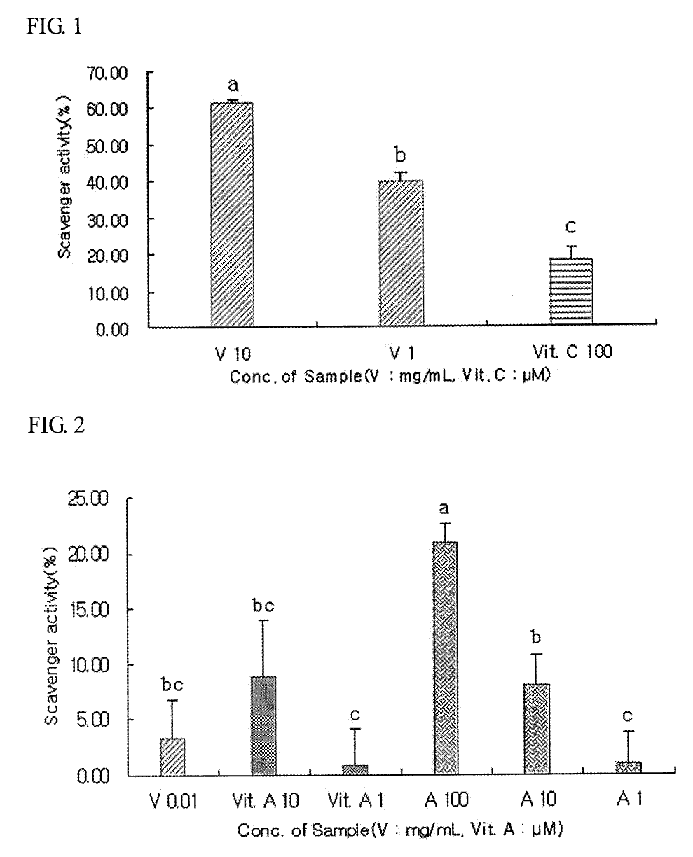

In the test results, the DPPH radical scavenger activity of the Vaccinium uliginosum extract was three times higher at a concentration of 10 mg/mL than 100 μM vitamin C, and about two times higher at a concentration of 1 mg/mL than 100 μM vitamin C (see FIG. 1). FIG. 1 shows the DPPH radical scavenger activity of the Vaccinium uliginosum extract prepared in Preparation Example 1. Alphabetic letters shown in the upper portion of FIG. 1 show values significantly different at p<0.05 among the groups by the Duncan's multiple range test.

2. Measurement of Superoxide Radical Scavenger Activity

2-1. Xanthine-Xanthine Oxidase System

On a 24-well plate, 600 μl of 0.1 M phosphate buffer (pH 7.5), 50 μl of each of Vaccinium uliginosum extract solutions having varying extract concentrations, and 50 μl of xanthine oxidase (0.068 μg/mL), were mixed with each other and left to stand at 25° C. for 30 minutes. To the mixture, 100 μl of 1 M HCL was added to stop the reaction and then measured for absorbance at 295 nm. As a control drug, allopurinol known as a drug functioning to inhibit xanthine oxidase was used. The results were expressed as percentages relative to the group untreated with the sample. As shown in FIG. 2, the activity of the Vaccinium uliginosum extract to scavenge superoxide radicals in the xanthine-xanthine oxidase system which is an enzymatic superoxide radical production system was the same as 10 μM vitamin A at a concentration of 0.01 mg/mL and corresponded to the scavenger activity between 1 μM allopurinol and 10 μM allopurinol.

2-2. NADH-PMS System

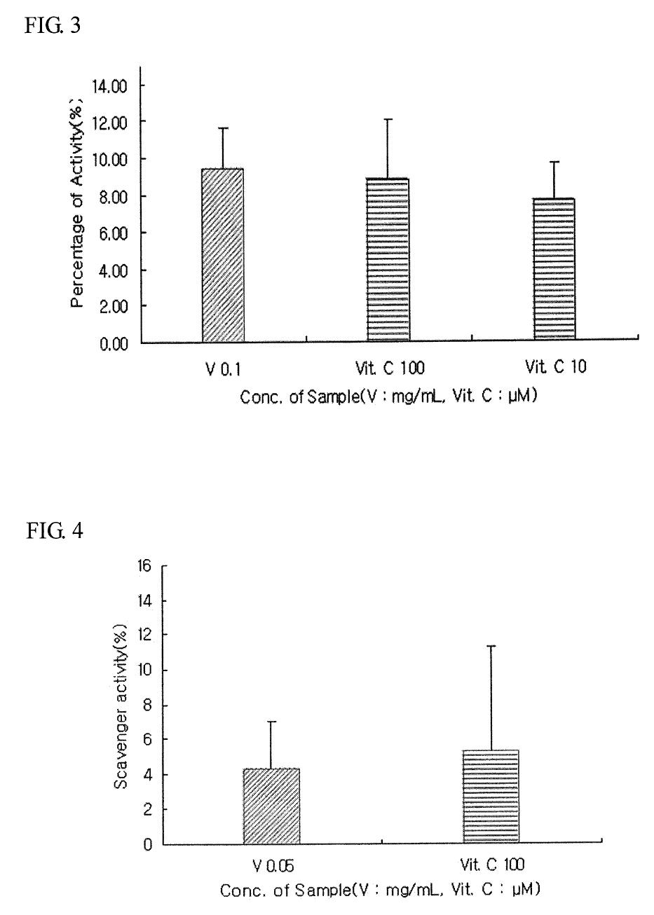

On a 24-well plate, NADH, phenazine methosulfate and NBT were added to 20 mM potassium phosphate buffer (pH 7.4) at concentrations of 73 μM, 15 μM and 50 μM, respectively, to prepare 1.8 mL of a solution. To the solution, 0.2 ml of the Vaccinium uliginosum extract prepared in Preparation Example 1 was added at varying concentrations. The mixture was left to stand at 37° C. for 20 minutes and then measured for absorbance at 560 nm. As a control drug, ascorbic acid (vitamin C) was used. The results were expressed as percentages relative to the group untreated with the sample. As shown in FIG. 3, the activity of the Vaccinium uliginosum extract to scavenge superoxide radicals in the NADH/PMS system which is a non-enzymatic superoxide radical production system was equal to that of 100 μM vitamin C at an extract concentration of 0.1 mg/mL.

3. Measurement of Hydroxyl Radical Scavenger Activity

On a 24-well plate, 0.8 ml of 5.94 mM H₂O₂ and 0.8 ml of ethanol solution containing 26.4 mM FeSO₄ were added to 0.2 ml of β-carotene ethanol solution, to which 0.2 ml of the Vaccinium uliginosum extract prepared in Preparation Example 1 was added. The mixture was measured for absorbance at 436 nm. As a control drug, ascorbic acid (vitamin C) was used. The results were expressed as percentages relative to the group untreated with the sample. The test results showed that the hydroxyl radical scavenger activity of the Vaccinium uliginosum extract was similar to that of 100 μM vitamin C at an extract concentration of 0.05 mg/mL (see FIG. 4).

4. Measurement of Singlet Oxygen Scavenger Activity

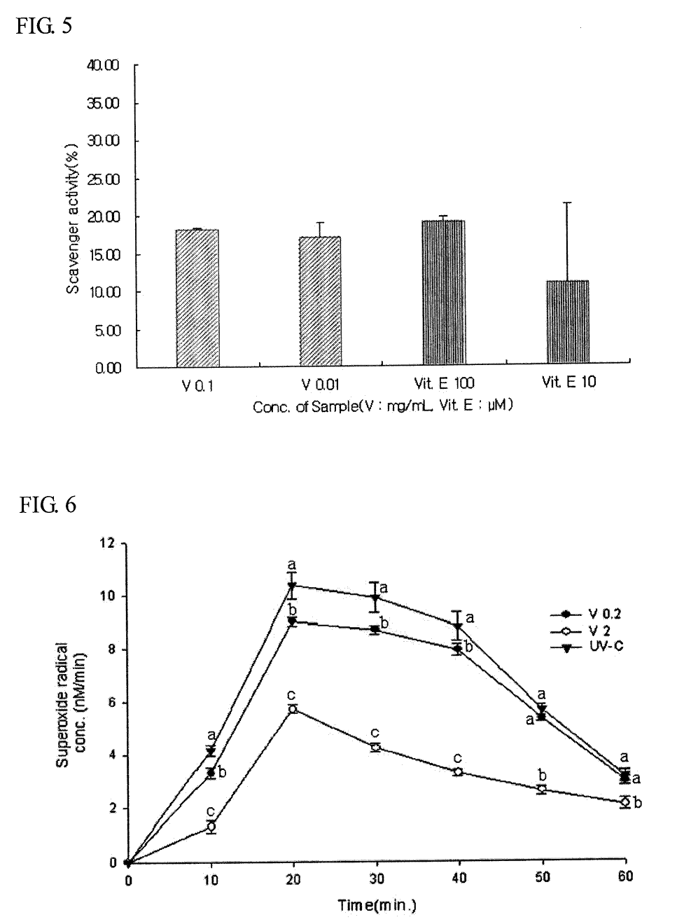

To 1.9 ml of a mixed solution of 10 mM histidine, 10 mM NaOCl, 10 mM H₂O₂ and 50 mM N,N-dimethyl-p-nitrosoaniline in 45 mM sodium phosphate buffer (pH 7.1), 0.1 ml of Vaccinium uliginosum extract was added at varying concentrations. The resulting solution was left to stand at 30° C. for 40 minutes and then measured for absorbance at 440 nm. As a control drug, α-tocopherol (vitamin E) was used. The results were expressed as percentages relative to the group untreated with the sample. The test results showed that the singlet oxygen scavenger activity of the Vaccinium uliginosum extract had no difference between concentrations of 0.1 mg/mL and 0.01 mg/mL and was equal to that of 100 μM vitamin E (see FIG. 5).

### Test Example 2

### Ability to Inhibit Production of ROS in Keratinocytes

Culture of Human Keratinocytes

Human keratinocytes were collected by biopsy from the skin tissue of a 13-year-old man and cultured in keratinocyte basal medium (modified MCDB 153 medium) containing 100 ng/mL of recombinant human epidermal growth factor, 70 mg/mL of bovine pituitary extract, 0.5 mg/mL of hydrocortisone, 5 mg/mL of insulin, 0.3 mg/mL of gentamicin and 2.5 mg/mL of amphotericin-B, in a CO₂ incubator in conditions of 37° C. and 5.0% CO₂. For use in tests, the keratinocytes were subcultured three times.

Measurement of Ability to Inhibit Production of Radicals in Keratinocytes

In order to measure ROS produced in keratinocytes when the skin is irradiated with ultraviolet radiation, keratinocytes were seeded into each well of a 24-well plate at a cell concentration of 10⁵ cells/well and left to stand for 17 hours, and the adhesion of the cells was confirmed. Then, the medium was removed and 2 ml of each of Vaccinium uliginosum extract solutions prepared by dissolving the extract in medium at varying concentrations was dispensed into each well of the plate and left to stand for 24 hours. After completion of the standing, the medium was removed and 400 μl of PBS (phosphate buffered saline) was dispensed into each well of the plate. Next, the solution in each well of the plate was irradiated with ultraviolet B radiation at a dose of 45 mJ/cm², and the amount of produced ROS was then measured at an interval of 10 minutes over 60 minutes.

1. Measurement of Ability to Inhibit Production of Superoxide Radicals

On a 24-well plate, 73 μM NADH, 15 μM phenazine methosulfate and 50 μM NBT were mixed with each other in 20 mM potassium phosphate buffer (pH 7.4) to prepare 1.8 ml of a solution, to which 0.2 ml of the Vaccinium uliginosum extract prepared in Preparation Example 1 was then added. 0.2 ml of the supernatant was collected at varying time points, left to stand at 37° C. for 20 minutes and then measured for absorbance at 560 nm.

The measurement results showed that, in the case of the group treated with 2 mg/ml of the Vaccinium uliginosum extract, the productions of superoxide radicals in keratinocytes after irradiation with ultraviolet radiation were 31%, 55%, 42%, 37%, 45% and 65% compared to the control group at 10 min, 20 min, 30 min, 40 min, 50 min and 60 min, and in the case of the group treated with 0.2 mg/mL of the Vaccinium uliginosum extract, the productions of superoxide radicals were 79%, 86%, 87%, 89%, 94% and 94% compared to the control group. During a period of time from 10 min to 40 min, the production of superoxide radicals was statistically significantly decreased in a concentration-dependent manner, and then, it was significantly decreased in the case of the group treated with 2 mg/ml of the Vaccinium uliginosum extract (see FIG. 6).

2. Measurement of Ability to Inhibit Production of Hydroxyl Radicals

On a 24-well plate, 0.8 ml of 5.94 mM H₂O₂, 0.8 ml of ethanol solution containing 26.4 mM FeSO₄, and 0.2 ml of the supernatant collected at each of time points were mixed with each other in 0.2 ml of 2.5 mM β-keratin ethanol solution. The resulting solution was measured for absorbance at 436 nm.

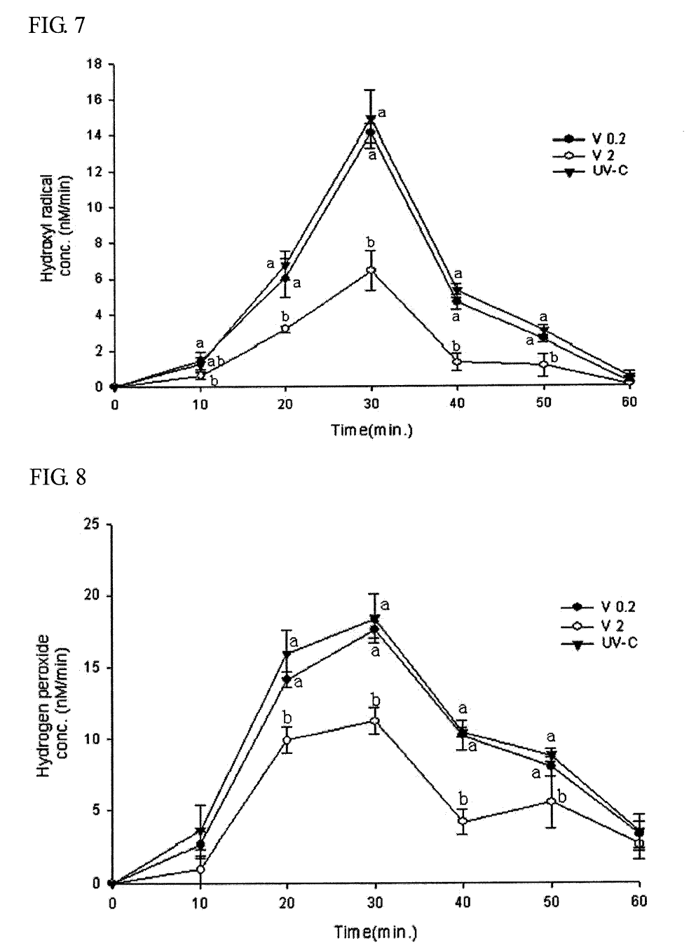

The measurement results showed that, in the case of the group treated with 2 mg/mL of the Vaccinium uliginosum extract, the production of hydroxyl radicals in keratinocytes after irradiation with ultraviolet B radiation was decreased over a period from 10 min to 50 min, and the percentages of production of hydroxyl radicals relative to the control group were 46%, 46%, 42%, 24% and 37% at 10 min, 20 min, 30 min, 40 min, 50 min and 60 min, respectively (see FIG. 7).

3. Measurement of Ability to Inhibit Production of Hydrogen Peroxide

1 ml of 3×10⁻⁶M scopoletin, 400 μl of 10⁻² M sodium azide and 0.5 mL of the supernatant collected at each of time points were mixed with each other and left to stand for 5 minutes. To the mixture, 100 μl of 150 U/mL HPO (Horseradish Peroxidase) and 600 μl of KRP (Kreps Ringer Phosphate buffer) were added, and the resulting solution was measured for absorbance using a spectrofluorometer under conditions of 360 nm excitation and 450 nm emission. The results are shown in FIG. 8. As shown in FIG. 8, the production of hydrogen peroxide in keratinocytes after irradiation with ultraviolet radiation was significantly reduced in the case of the group treated with the 2 mg/mL of the Vaccinium uliginosum extract compared to the control group untreated with the Vaccinium uliginosum extract, in which the percentages of production of hydrogen peroxide relative to the control group were 61%, 61%, 39% and 62% at 20 min, 30 min. 40 min and 50 min, respectively.

4. Measurement of Ability to Inhibit Production of Singlet Oxygen

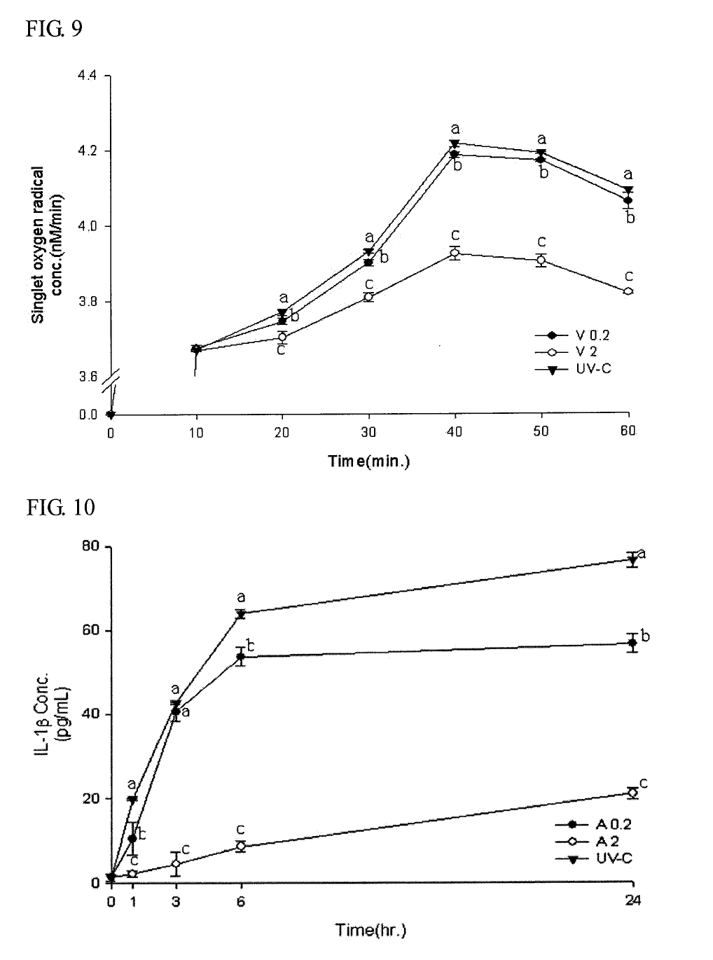

0.2 ml of the supernatant collected at each of time point was mixed with a solution of 10 mM histidine, 10 mM NaOCl, 10 mM H₂O₂ and 50 mM N,N-dimethyl-p-nitrosoaniline in 45 mM sodium phosphate buffer (pH 7.1) and left to stand at 30° C. for 40 minutes and then measured for absorbance at 440 nm. The results are shown in FIG. 9. In the case of the group treated with the Vaccinium uliginosum extract, the production of singlet oxygen radicals in keratinocytes after irradiation with ultraviolet B radiation was reduced starting from 20 minutes in a concentration-dependent manner. In comparison with the control group (UV-C), the group treated with the 2 mg/mL of the Vaccinium uliginosum extract (V 2) showed productions of 98% at 20 min, 96% at 30 min and 93% in a period of time from 40 min to 60 min, and the group treated with 0.2 mg/mL of the Vaccinium uliginosum extract (V 0.2) showed a production of 99%.

### Test Example 3

### Ability to Inhibit Secretion of Cytokines in Keratinocytes

In order to measure the amount of cytokines produced in keratinocytes when the skin is irradiated with ultraviolet radiation, keratinocytes were seeded onto a 24-well plate at a cell concentration of 10⁵ cells/well and left to stand for 17 hours, and the adhesion of the cells was confirmed. Then, the medium was removed and 2 ml of each of Vaccinium uliginosum extract solutions prepared by dissolving the extract in medium at varying concentrations was dispensed into each well of the plate, followed by standing for 24 hours. After completion of the standing, the medium was removed and 400 μl of PBS (phosphate buffered saline) was dispensed into each well of the plate, and the solution in each well was irradiated with UV (ultraviolet) B radiation at a dose of 40 mJ/cm². Then, the amount of cytokines produced in each solution was measured at varying time points for 24 hours.

1. Measurement of Ability to Inhibit Secretion of IL-1β

Keratinocytes were irradiated with 40 mJ/cm² of UV B, and after 0 hr, 30 min, 1 hr, 3 hr, 6 hr and 24 hr, the supernatant was collected and the amount of IL-1β was measured using an ELISA assay kit. The measurement results showed that the production of IL-1β in keratinocytes treated with 2 mg/mL of the Vaccinium uliginosum extract was significantly reduced compared to the control group, in which the percentages of production of IL-1β relative to the control group were about 37%, 28%, 29% and 26% at 1 hr, 3 hr, 6 hr and 24 hr, respectively. Also, the productions of IL-1β in keratinocytes treated with 0.2 mg/mL of the Vaccinium uliginosum extract were about 85%, 88%, 89% and 73% relative to the control group (see FIG. 10).

2. Measurement of Ability to Inhibit Secretion of IL-6

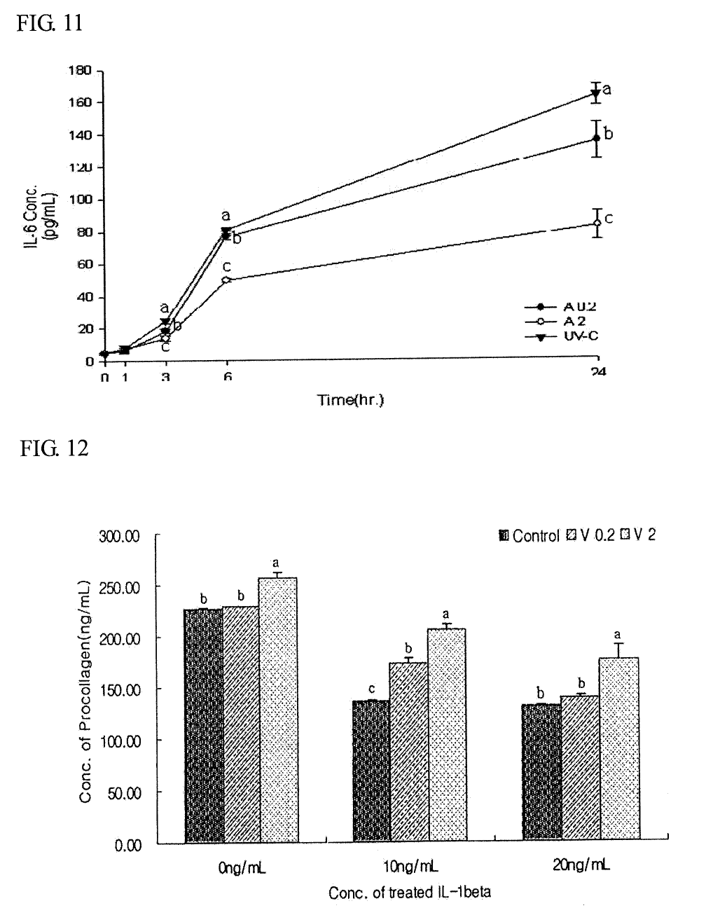

Keratinocytes were irradiated with 40 mJ/cm² of UV B, and after 0 hr, 1 hr, 3 hr, 6 hr and 24 hr, the supernatant was collected and the amount of IL-6 was measured using an ELISA assay kit. The test results showed that the production of IL-6 in keratinocytes treated with 2 mg/mL of the Vaccinium uliginosum extract was significantly reduced starting from 3 hours, and the percentages of production of IL-6 relative to the control group were 43%, 61% and 33% at 3 hr, 6 hr and 24 hr, respectively. Also, the group treated with 0.2 mg/mL of the Vaccinium uliginosum extract showed a significant reduction in the production of IL-6 at 6 hr, in which the percentage of production relative to the control group was about 81% (see FIG. 11).

### Test Example 4

### Test of Wrinkle Improvement Effect

Culture of Human Fibroblasts

Human fibroblasts were collected by biopsy from the skin tissue of a 13-year-old man and cultured in DMEM (Dulbecco's modified Eagle's medium) containing 10% fetal bovine serum, 100 IU/mL of penicillin and 50 μg/mL of streptomysin, in an incubator under conditions of 37° C. and 5.0% CO₂.

Test of Wrinkle Improvement Effect

Human fibroblasts were seeded into a 24-well plate at a cell concentration of 10⁵ cells/well, and after 17 hours, the adhesion of the cells was confirmed. Then, the medium was removed and 2 ml of each of Vaccinium uliginosum extract solutions prepared by dissolving the extract of Preparation Example 1 in medium at varying concentrations was dispensed into each well of the plate. The well plate was incubated in a CO₂ incubator for 48 hours, and then the medium was removed and type I procollagen (MMP-1) in the human fibroblasts was measured.

1. Promotion of Type I Procollagen in Human Fibroblast Cells

Human fibroblasts were treated with varying concentrations of Vaccinium uliginosum extract solutions, and after 48 hours, the supernatant was collected and then the amount of type I procollagen produced in the human fibroblasts was measured using the procollagen type I C-Peptide (PIP) EIA kit.

TABLE 3

| Concentration of sample (mg/ml) | Concentration of procollagen (ng/ml) |
|---|---|
| Control | 149.6 ± 25.4¹⁾ |
| 0.001 | 144.9 ± 21.8 |
| 0.005 | 155.1 ± 20.1 |
| 0.01 | 179.2 ± 31.3 |

¹⁾Mean ± S.D.

The test results showed that the productions of type I procollagen in human fibroblasts treated with the Vaccinium uliginosum extract were 155.1 ng/mL (103%) at 0.005 mg/mL and 179.2 ng/mL (119%) at 0.01 mg/mL as compared to 149.6 ng/mL for the control group untreated with the inventive extract. This indicates that the production of type I procollagen in human fibroblasts treated with the inventive extract was increased in a concentration-dependent manner. Collagen fibers are formed by a process wherein procollagen is synthesized in fibroblasts and secreted out of the cells and then subjected to enzymatic action, fiber formation and crosslinking. Accordingly, it was found that the Vaccinium uliginosum extract can increase the amount of procollagen, a precursor of collagen, thus enhancing skin firmness and improving wrinkles.

2. Inhibition of Matrix Metalloproteinase-1 (MMP-1) Produced

Human fibroblasts were treated with the Vaccinium uliginosum extract solutions at varying extract concentrations as shown in Table 4 below. After 48 hours, the supernatant was collected and the amount of matrix metalloproteinase-1 (MMP-1) produced in the fibroblasts was measured using the MMP-1 EIA kit.

TABLE 4

| Concentration of sample (mg/ml) | Concentration of MMP-1 (ng/ml) |
|---|---|
| Control | 37.2 ± 4.7¹⁾ |
| 0.001 | 32.8 ± 3.1 |
| 0.005 | 27.1 ± 5.5 |
| 0.01 | 25.5 ± 1.3 |

¹⁾Mean ± S.D.

In the test results, the amount of biosynthesis of MMP-1 in the control group untreated with the inventive extract was 37.2 mg/mL, whereas the production of MMP-1 in the group treated with the Vaccinium uliginosum extract was 25.5 ng/mL (68%) at 0.01 mg/mL and reduced in a concentration-dependent manner. It is known that MMP-1 is expressed in keratinocytes and fibroblasts by repeated exposure to ultraviolet radiation and is an enzyme that decomposes collagen. Accordingly, it was found that the Vaccinium uliginosum extract could inhibit the activity of MMP-1 to inhibit the decomposition of skin collagen, thus enhancing skin firmness and improving wrinkles.

3. Change in Production of Type I Procollagen Upon Addition of IL-1β to Human Fibroblasts

It can be seen that the amount of type I procollagen produced upon the addition of interlenkin (IL)-1β to human fibroblasts is lower than the amount produced when IL-1β is no added, and particularly, the amount of production of type I procollagen is reduced in a manner dependent on the concentration of IL-1β. However, it could be found that, when the Vaccinium uliginosum extract was added, the production of type I procollagen would be increased in a manner dependent on the concentration of the Vaccinium uliginosum extract. FIG. 12 shows the production of type I procollagen in human fibroblasts as a function of IL-1β concentrations (10 ng/mL and 20 ng/mL) and Vaccinium uliginosum extract concentrations (0.2 mg/mL and 2 mg/mL).

4. Change in Production of MMP-1 Upon Addition of IL-1β to Human Fibroblasts

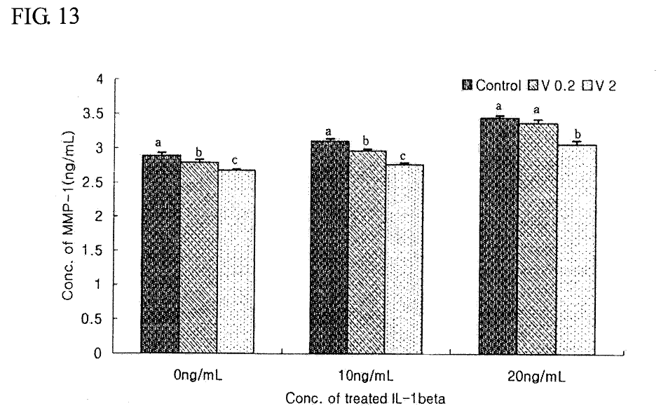

As shown in FIG. 13, it can be seen that the amount of MMP-1 produced upon the addition of interleukin (IL)-1β to human fibroblasts is higher than the amount produced when IL-1β is not added, and the production of MMP-1 is increased in a manner dependent on the concentration of IL-1β. However, even in this case, it could be found that, when the Vaccinium uliginosum extract was added, the concentration of MMP-1 would be reduced in a manner dependent on the concentration of the Vaccinium uliginosum extract.

### Test Example 5

### Test of Ability to Inhibit Formation of Wrinkles In Vivo

Hairless Mice, Ultraviolet Irradiation and Sample Administration

6-week-old female hairless mice (Skh-1) were purchased and acclimated for 3 days after reaching the laboratory and then used in tests. The animals were allowed to freely eat food and water and bred under conditions of temperature 24±2° C., humidity of 50±10% and a 12-hr day/12-hr night cycle. The animals were divided into a group irradiated with ultraviolet radiation, a group non-irradiated with ultraviolet radiation and a group irradiated with ultraviolet radiation and administered with a sample. The group irradiated with ultraviolet radiation and administered with a sample was administered with the Vaccinium uliginosum extract at each of doses of 10, 20 and 40 mg/kg.

Ultraviolet radiation was irradiated on the back of each of the mice three times a week for 18 weeks, in which the doses of ultraviolet radiation were 1 MED (minimal erythromal dose; 60 mJ/cm²) for the first week, 2 MED (120 mJ/cm²) for the second and third weeks, 3 MED (180 mJ/cm²) for the fourth to sixth weeks), and 4 MED (240 mJ/cm²) for the seventh to eighteen weeks. The Vaccinium uliginosum extract as the sample was dissolved in distilled water and administered to the animals at each of doses of 10, 20 and 40 mg/kg/day.

Fabrication of Skin Replica

In order to measure wrinkle improvement caused by administration of the sample to the mice, 50 mg/kg of pentobarbital solution was intraperitoneally injected at an interval of 3 weeks to anesthetize the mice, and a replica mold having a hole diameter of 8 mm was attached to the back of each of the mice. Then, the components of Silflo (i.e., silicone rubber, a thinner and a catalyst) were suitably blended with each other, applied to the inner side of the mold and naturally dried. Then, the mold was detached, thus fabricating a replica. The replica was irradiated with light at a constant angle through a computer imaging system, and the area of the resulting reflection was used to quantify the depth or number of wrinkles and to measure the degree of wrinkles.

Results of Skin Replica Analysis

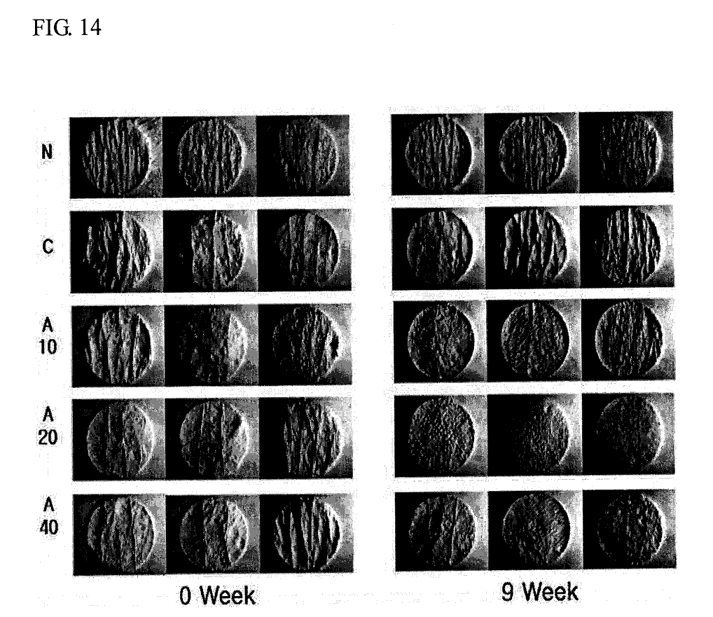

In visual observation after ultraviolet irradiation, an increase in skin wrinkles in the UV control group (C) compared to the normal group (N) was clearly shown. However, in case of the skin administered with the Vaccinium uliginosum extract, a reduction in thick wrinkles was clearly shown at 9 weeks after the administration (see FIG. 14).

At 3 weeks after the administration, the groups administered with the Vaccinium uliginosum extract in amounts of 20 mg/kg (V 20) and 40 mg/kg (V 40) showed significant reductions in H_R 1, 4 and 5 values compared to the UV control group (C). At 6 weeks after the administration, all values for all the groups administered with the inventive extract were significantly reduced compared to the UV control group, except that the group administered with 20 mg/kg of the Vaccinium uliginosum extract showed reductions in H_R 2 and 3 values. At 9 weeks after the administration, H_R values in the groups administered with the Vaccinium uliginosum extract were all significantly reduced.

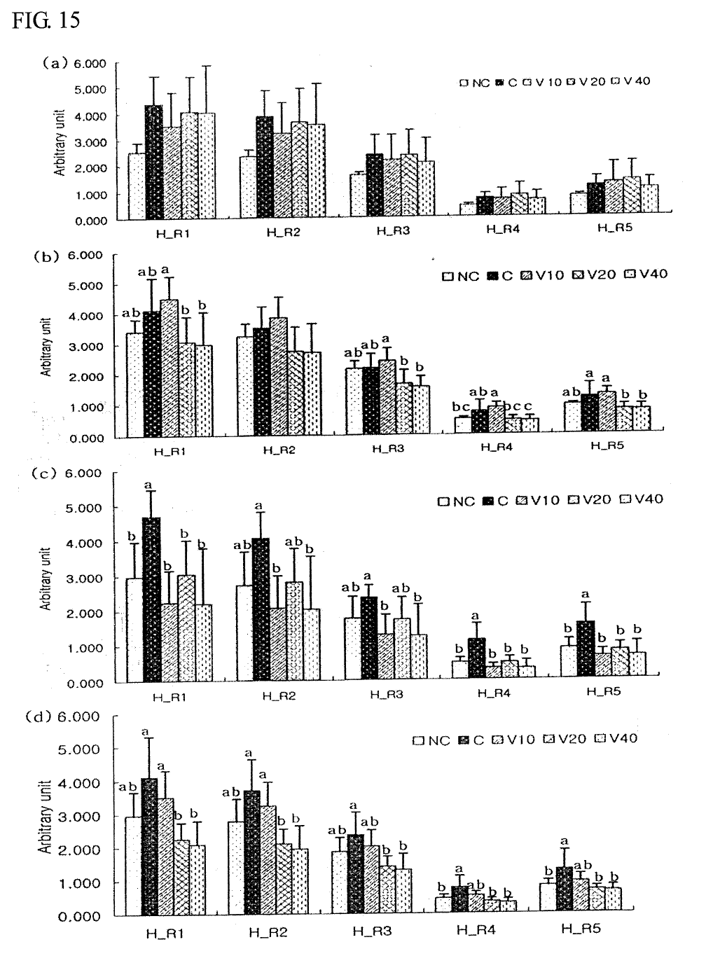

FIGS. 15(a) to 15(d) show H_R values at 0, 3, 6 and 9 weeks after ultraviolet irradiation. Herein, “H” means horizontal, “R1” represents a distance between the highest mountain and the lowest value, “R2” represents the greatest value of those five maximum distances, “R3” represents the average of five maximum distance R1, R4 represents smoothness depth, and “R5” represents arithmetic average roughness. Letters (alphabets) different superscripts of FIG. 15 are significantly different at p<0.05 among the Duncan's multiple range test.

### Test Example 6

### Inhibitory Effect on Tyrosinase Activity

To a 96-well plate (Corning, USA), 220 μl of 0.1 M PBS (pH 6.5), 20 μl of each of Vaccinium uliginosum extract solutions having different extract concentrations, and 20 μl of 2,000 U/mL tyrosinase solution, were sequentially added. To the solution, 40 μl of 1.5 mM tyrosine solution was added, followed by standing at 37° C. for 10 minutes. Then, the solution was measured for absorbance at 490 nm using an enzyme-linked immunosorbent assay, ELISA) reader (Bio-Tek, USA). As a blank sample, 20 μl of 0.1 M PBS (pH 6.5) was used in the test. As a control group, a group treated with kojic acid was used. The inhibition rate of tyrosinase activity is calculated according to the following equation.

Inhibition rate (%) of tyrosinase activity=100−{(b-b′)/(a-a′)}×100

wherein a: absorbance after reaction of blank sample; b: absorbance after reaction of sample; and a′ and b′: absorbance measured using buffer in place of tyrosinase in reaction of each sample.

In the test results, the Vaccinium uliginosum extract showed an IC₅₀ of about 0.41 mg/mL against tyrosinase activity, and kojic acid showed a high IC₅₀ of about 10 μM. The Vaccinium uliginosum extract showed an inhibition rate of 72.8% against tyrosinase activity at a concentration of 1 mg/mL, and this inhibition rate was between 50.8% at 10 μM of kojic acid and 84.8% at 100 μM of kojic acid. Also, the Vaccinium uliginosum extract showed an inhibition rate of 11.4% at a low concentration of 0.1 mg/mL, and this inhibition rate was similar to an inhibition rate of 14.4% at 1 μM of kojic acid (see Table 5).

TABLE 5

| | Concentration of sample | Inhibition rate (%) |
|---|---|---|
| Kojic acid (μM) | 100 | 84.4 ± 8.1¹⁾²⁾ |
| | 10 | 50.8 ± 9.6 |
| | 1 | 14.4 ± 4.5 |
| | 0.1 | 9.7 ± 3.7 |
| Vaccinium uliginosum L. | 1 | 72.8 ± 5.7 |
| (mg/ml) | 0.5 | 58.4 ± 3.4 |
| | 0.25 | 33.2 ± 4.8 |
| | 0.1 | 11.4 ± 2.4 |

¹⁾Mean ± S.D.
²⁾Inhibition rate (%)

Although the direct comparison between the two substances was impossible due to different concentrations, it can be seen from the test results that 0.5 mg/ml of the Vaccinium uliginosum extract was higher in the inhibition rate of tyrosinase activity than that of the group treated with 10 μM of kojic acid, suggesting that the Vaccinium uliginosum extract significantly inhibited tyrosinase activity. Tyrosinase functions to oxidize tyrosine (a kind of amino acid) to produce melanin, and from the above test results showing that the Vaccinium uliginosum extract inhibits tyrosinase activity, it can be seen that the Vaccinium uliginosum extract can enhance skin whitening.

### Test Example 7

### Test of Inhibitory Effect on Melanin Production Using B 16 Melanoma F 10 cells

Culture of B16 melanoma F10

B16 melanoma F10 cells were cultured in DMEM (Dulbecco's modified Eagle's medium) containing 10% fetal bovine serum, 100 IU/mL of penicillin and 50 μg/mL of streptomysin, in a CO₂ incubator under conditions of 37° C. and 5.0% CO₂.

Test of Inhibitory Effect on Melanin Production

B 16 melanoma F 10 cells were seeded into each well of a 24-well plate at a cell concentration of 10⁴ cells/well. After 17 hours, the adhesion of the cells was confirmed and the medium was removed. Then, 2 ml of the Vaccinium uliginosum extract solution prepared in Preparation Example 1 was dispensed into each well of the plate at varying concentrations. The plate was incubated in a CO₂ incubator for 72 hours and then the medium was removed. Next, 2 ml of NaOH was added into each well of the plate, followed by standing at 60° C. for 30 minutes. Then, the cell solution was measured for absorbance at 450 nm. The concentration of melanin was calculated compared to the melanin standard curve.

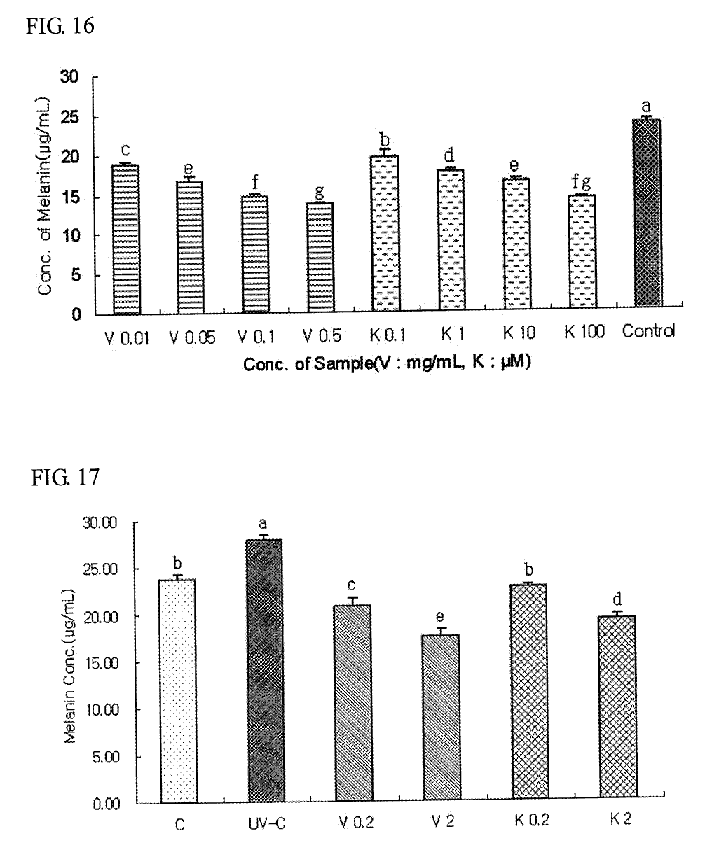

In the test results, total melanin production in B 16 melanoma F10 cells treated with the Vaccinium uliginosum extract was significantly reduced in a concentration-dependent manner over a concentration range from 0.01 mg/mL to 0.5 mg/mL. The production of melanin at a Vaccinium uliginosum extract concentration of 0.05 mg/mL was 16.66 μM/mL, and the production of melanin at 10 μM kojic acid was 16.54 μg/mL, indicating that the melanin production inhibitory effect at 0.05 mg/mL of the Vaccinium uliginosum extract was the same as the melanin production inhibitory effect at 10 μM kojic acid. Also, the melanin production at 0.5 mg/mL of the Vaccinium uliginosum extract was lower than the melanin production at 100 μM kojic acid (see FIG. 16). In FIG. 16, “V” represents a test group treated with the Vaccinium uliginosum extract, and “K” represents a test group treated with kojic acid.

Test of Inhibitory Effect on Total Melanin Production Upon Ultraviolet Irradiation

Melanin productions in groups of B16 melanoma F10 cells treated with the Vaccinium uliginosum extract solutions having extract concentrations of 0.2 mg/mL and 2 mg/mL were 20.80 μg/mL and 17.47 μg/mL which were lower than that of the control group (27.96 μg/mL), and the melanin production was reduced in a manner dependent on the concentration of the Vaccinium uliginosum extract. Also, the melanin production at 0.2 mg/mL of the Vaccinium uliginosum extract was lower than that of 0.2 μM kojic acid (22.72 μg/mL), and the melanin production at 2 mg/mL of the Vaccinium uliginosum extract was lower than that of 2 μM kojic acid (19.22 μg/mL) (see FIG. 17). In FIG. 17, “C” represents a test group non-irradiated with ultraviolet radiation, “UV-C” represents a control group irradiated with ultraviolet radiation, and “V” and “K” represent test groups treated with the Vaccinium uliginosum extract and kojic acid, respectively.

In the test results, the group treated with the Vaccinium uliginosum extract and the group treated with kojic acid all showed a reduction in melanin production compared to the control group untreated with the sample. Pigmentations occurring on the skin, such as discoloration and freckles, are attributable to the abnormal increase of melanin pigment in the epidermis. Although the Vaccinium uliginosum extract and the kojic acid all showed a significant reduction in melanin production, the kojic acid has the problem of toxicity. Considering this problem, it can be found that the Vaccinium uliginosum extract is useful as a substitute for the kojic acid.

Although preferred embodiments of the present invention have been described for illustrative purposes, those skilled in the art will appreciate that various modifications, additions and substitutions are possible, without departing from the scope and spirit of the invention as disclosed in the accompanying claims.

### INDUSTRIAL APPLICABILITY

As described above, the inventive composition for skin whitening and wrinkle improvement can be easily prepared in an extract or dried extract powder and can be used for the enhancement of skin whitening, the removal of wrinkles, the prevention of wrinkles and the increase of skin firmness. Also, the inventive composition is based on the natural extract, and there is no particular limitation on the industrial utilization range of the inventive composition. Furthermore, the Vaccinium uliginosum extract is suitable to use as a component for improving skin wrinkles in cosmetics, foods and drugs, because the Vaccinium uliginosum extract can achieve the effect of improving skin conditions, even when it is applied to the skin or used internally.

1. A composition for skin whitening and wrinkle improvement, which comprises a Vaccinium uliginosum extract as an active ingredient and has the effects of scavenging and inhibiting the formation of reactive oxygen species produced in skin tissue as a result of ultraviolet irradiation to the skin.

2. The composition of claim 1, wherein the Vaccinium uliginosum extract is extracted by adding water or alcohol as an extraction solvent to the fruit or leaf of Vaccinium uliginosum.

3. The composition of claim 1, wherein the Vaccinium uliginosum extract is obtained by heating the fruit or leaf of Vaccinium uliginosum in a water bath at a temperature of 40-100° C. to obtain a hot water extract, filtrating the hot water extract, separating the supernatant from the filtered hot water extract, and concentrating the separated supernatant under reduced pressure.

4. The composition of claim 1, wherein the Vaccinium uliginosum extract is obtained by extracting the fruit or leaf of Vaccinium uliginosum in alcohol at a temperature of 20-90° C., filtering the extract, separating the supernatant from the filtered extract, and concentrating the separated supernatant under reduced pressure.

5. A cosmetic composition for skin whitening and wrinkle improvement, which comprises the composition of any one of claims 1 to 4 and cosmetic additives and has the effects of whitening the skin and improving wrinkles.

6. The cosmetic composition of claim 5, wherein the content of the Vaccinium uliginosum extract is 0.0001-10% by dry weight based on the total weight of the cosmetic composition.

7. The cosmetic composition of claim 5, which additionally comprises at least one component selected from the group consisting of arbutin, kojic acid, a Broussonetia extract, 3-ethoxy ascorbic acid, a licorice extract, and a mixture thereof.

8. A food composition for skin whitening and wrinkle improvement, which comprises the composition of any one of claims 1 to 4 and sitologically acceptable food additives.

9. A pharmaceutical composition for skin whitening and wrinkle improvement, which comprises the composition of any one of claims 1 to 4 and a pharmaceutically acceptable carrier and has the effects of increasing the synthesis of collagen and inhibiting tyrosinase activity.

10. A method for preparing a Vaccinium ulginosum extract for skin whitening and wrinkle improvement, comprising the steps of:
(a) adding water or alcohol as a solvent to the fruit or leaf of Vaccinium ulginosum to obtain a Vaccinium ulginosum extract;
(b) filtering the Vaccinium ulginosum extract obtained in the step (a) and separating the supernatant from the filtrate; and
(c) concentrating the supernatant separated in the step (b).

11. The method of claim 10, which further comprises, following the step (c), (d) dissolving the concentrated Vaccinium uliginosum extract in a small amount of distilled water and freeze-drying or drying the solution to obtain a powdery Vaccinium uliginosum extract.
